(19) United States
(12) Patent Application Publication
(10) Pub. No.: US 2016/0151148 A1
(43) Pub. Date: Jun. 2, 2016

(54) INSERTION OF AN ARTIFICIAL LIGAMENT

(71) Applicant: LOCKDOWN MEDICAL LIMITED, Redditch, Worcester-shire (GB)

(72) Inventors: Angus WALLACE, Nottingham, Nottinghamshire (GB); Sunil DHAR, Nottingham, Nottinghamshire (GB)

(73) Assignee: Lockdown Medical Limited, Redditch, Worcestershire (GB)

(21) Appl. No.: 14/904,282

(22) PCT Filed: Jul. 8, 2014
(86) PCT No.: PCT/GB2014/052073

§ 371 Date: Jan. 11, 2016

(30) Foreign Application Priority Data
Jul. 11, 2013 (GB) 1312466.4

(51) Int. Cl. A61F 2/08

(52) U.S. Cl. CPC A61F 2/0811; A61F 2002/0852

(57) ABSTRACT

The present invention provides a method for the insertion of an artificial ligament into an ankle of a human. The method comprises drilling a first hole through the talar neck of the ankle, and drilling a second hole though the distal part of the fibular. The method further comprises passing the artificial ligament through the first hole, looping the artificial ligament over the dorsal part of the talus neck and using a soft loop of the artificial ligament to anchor the artificial ligament over the talar neck. The artificial ligament is then passed through the second hole and securing the artificial ligament onto the calcaneus using a screw inserted through a hard look of the artificial ligament.

The present invention relates to a medical procedure for the insertion of an artificial ligament into a human or animal body and relates particularly, but not exclusively, to such a procedure for repairing or strengthening a natural ligament in, for example, the ankle joint and subtalar joints of a human being.

It is known to provide artificial replacement or supplemental ligaments in the human or animal body so as to repair or augment damaged or diseased ligaments. One such device is available from Lockdown Medical Limited and sold under the Lockdown™ brand. Such a device includes a woven or braded structure having a loop at each end and including a mid-portion of load carrying, generally elongate structure which replaces or supplements the mechanical security provided by a natural ligament. The loops are used in the securing process and, generally, a hard loop is used in association with a fixation device such as a screw which secures the ligament to a portion of bone and a soft loop is used to allow the creation of a loop in the artificial ligament itself which may be used to secure the other end to a different portion of bone structure. The present invention applies this ligament to in an otherwise unknown procedure which provides a new method of securing or stabilising the ankle and/or subtalar joints.

The present invention provides a medical repair or replacement procedure for ligaments in the ankle region. Such procedures may be used in advance of ligament damage or after ligament damage and may be used to supplement or even replace a ligament. The process is a method of treatment to correct or supplement ligaments in the ankle and is a repair system or augmentation system directed to the ligament discussed herein. Such an ankle ligament repair or supplemental procedure may be used to strengthen a weakened ankle ligament structure in sportsmen and non-sportsmen in advance of ligament damage and will find equal application amongst the normal population.

Accordingly, the present invention provides a method for the insertion of an artificial ligament into an ankle of a human. The method comprises drilling a first hole through the talar neck of the ankle, and drilling a second hole though the distal part of the fibular. The method further comprises passing the artificial ligament through the first hole, looping the artificial ligament over the dorsal part of the talus neck and using a soft loop of the artificial ligament to anchor the artificial ligament over the talar neck. The artificial ligament is then passed through the second hole before securing the artificial ligament onto the calcaneus using a screw inserted through a hard loop of the artificial ligament.

Preferably, the method further includes the step of using a measuring device that has been passed through the first hole and the second hole to determine the length of the artificial ligament to be used.

Advantageously, the measuring device is used to pull the artificial ligament through the first hole and the second hole.

Preferably, first hole is drilled using a cannulated drill bit. The bit may be a 5.5 mm bit.

Preferably, the second hole is drilled through the distal part of the fibula, in the anteroposterior (AP) direction, approximately 7 mm proximal from the tip of the fibula.

Advantageously, the second hole is drilled using a cannulated drill bit. The bit may be a 5.5 mm bit.

The method may further comprise the step of initially making a longitudinal, ventrally curved skin incision just over the posterior margin of the lateral malleolus.

The method may further comprise the steps of making an incision on the medial side of the talus adjacent to the first hole.

The invention also provides a kit of parts for use in the method of any one of claims 1 to 8 and including one or more artificial ligaments having an elongate portion, a first end including a loop for receiving a securing member and a second end including a loop for allowing said elongate portion to be passed therethrough such as to form a loop for securing to bone material and one or more securing members.

Conveniently, the kit of parts may include a plurality of artificial ligaments of different lengths.

The kit of parts may also include a plurality of bone securing members in the form of screws or bollards.

The kit may also include a length gauge.

In the broadest application of the present invention there is provided a method for the insertion of an artificial ligament having a first end and as second end into the human or animal body comprising the steps of: drilling a first hole A through a first portion of bone material such as to provide an anchoring point, drilling a second hole B through a second, different, portion of bone material which it is desired to secure, securing the first end of the ligament to the anchor point, passing the second end of the ligament through said second hole and tensioning said ligament before securing said second end to a further portion of bone material separate from said first portion.

Preferably, the ligament is provided having a soft loop at said first end and said ligament is secured to said anchor point by passing the second end of said ligament through said hole A and then passing said second end through the soft loop of said first end.

Advantageously, the ligament is provided with a hard loop at said second end and said second end is secured to said further portion of bone material by a fixation device passing through said hard loop.

Preferably, the method includes the step of securing said hard loop end to said further portion of bone material by inserting a screw or bollard through said hard loop and securing said screw or bollard to said further portion of bone material.

In the above process the first portion of bone material may be the talar neck T of an ankle, the second portion of bone material may be the distal part of the fibular and the further portion of bone material may be the calcaneus.

The present invention will now be more particularly described by way of example only with reference to the accompanying drawings, in which:

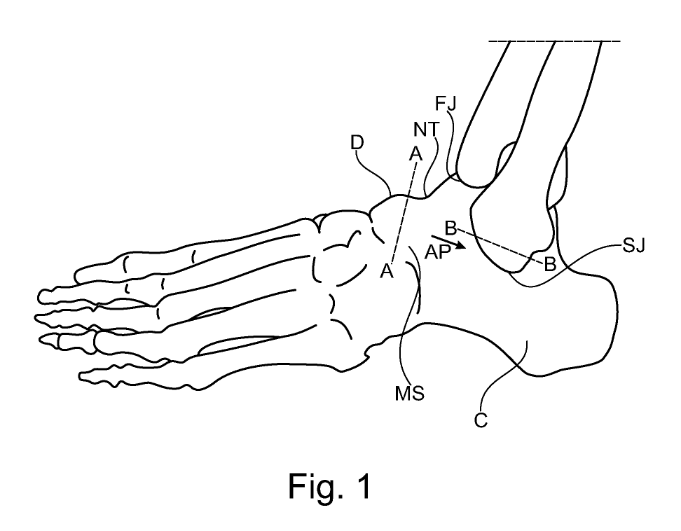

FIG. 1 is a lateral view of the bones of the ankle before an artificial ligament is fixed in place and illustrates the preferred orientations of the required holes;

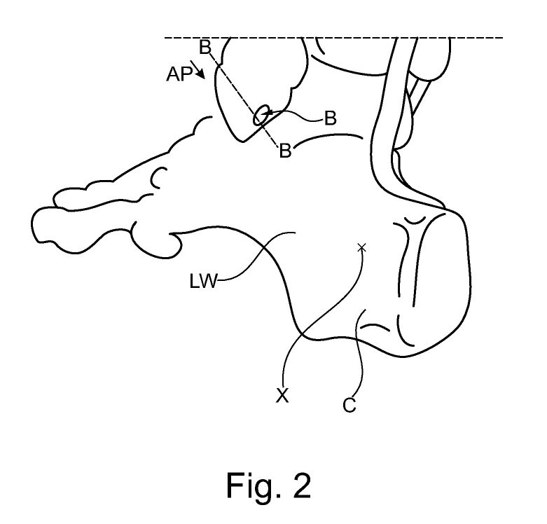

FIG. 2 is a close-up lateral view of the ankle before the artificial ligament is fixed in place and also illustrates the preferred angular orientation of one of the required holes and the location for the positioning of a fixation screw or bollard;

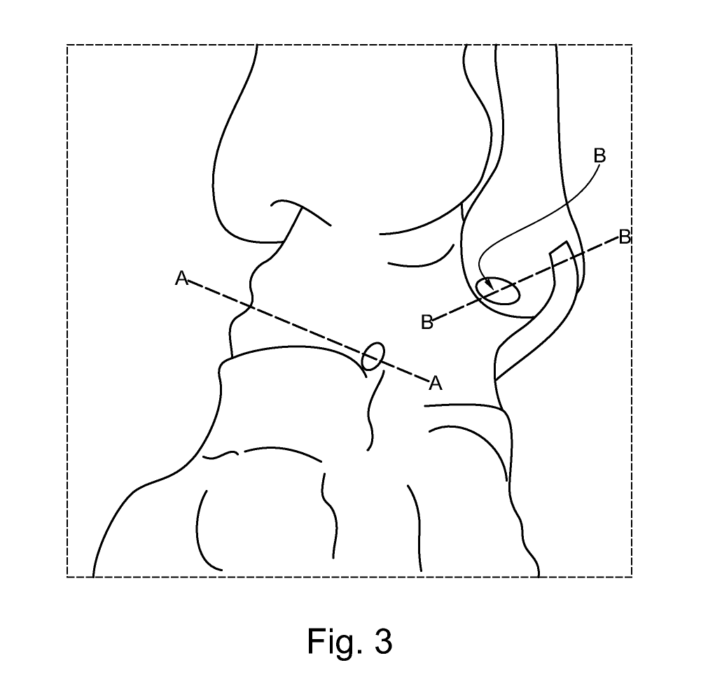

FIG. 3 is a close-up lateral view of the bones of the ankle showing the holes drilled in their desired positions;

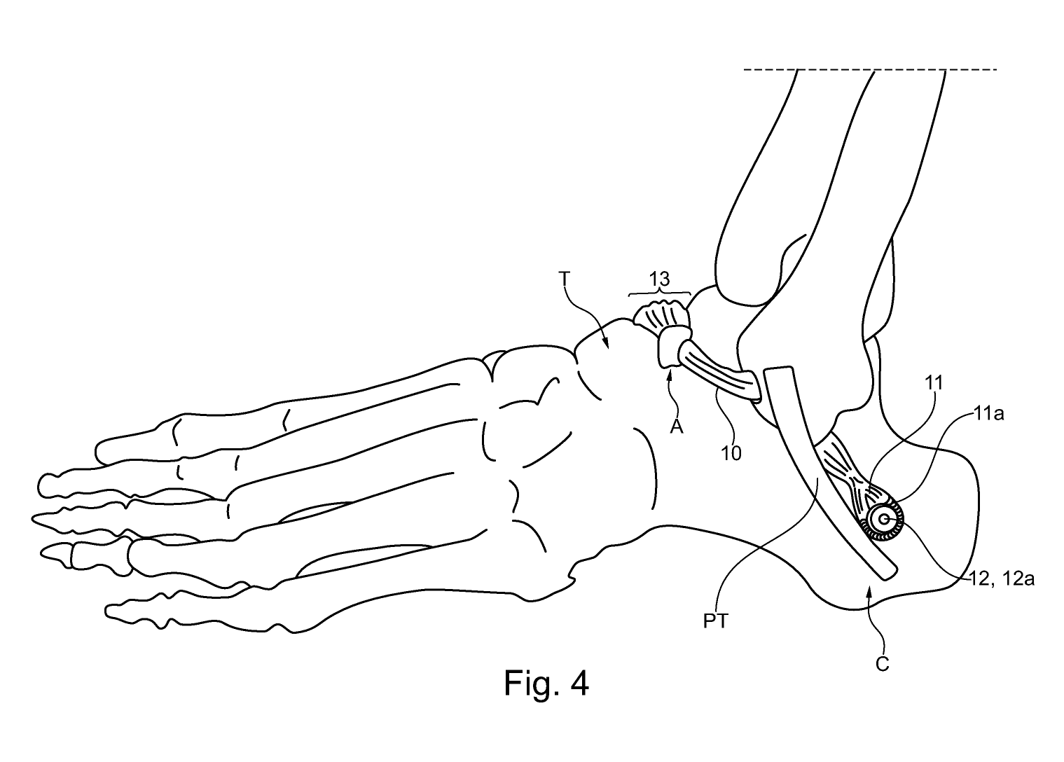

FIG. 4 is a lateral view of the bones of the ankle with an artificial ligament fixed in place;

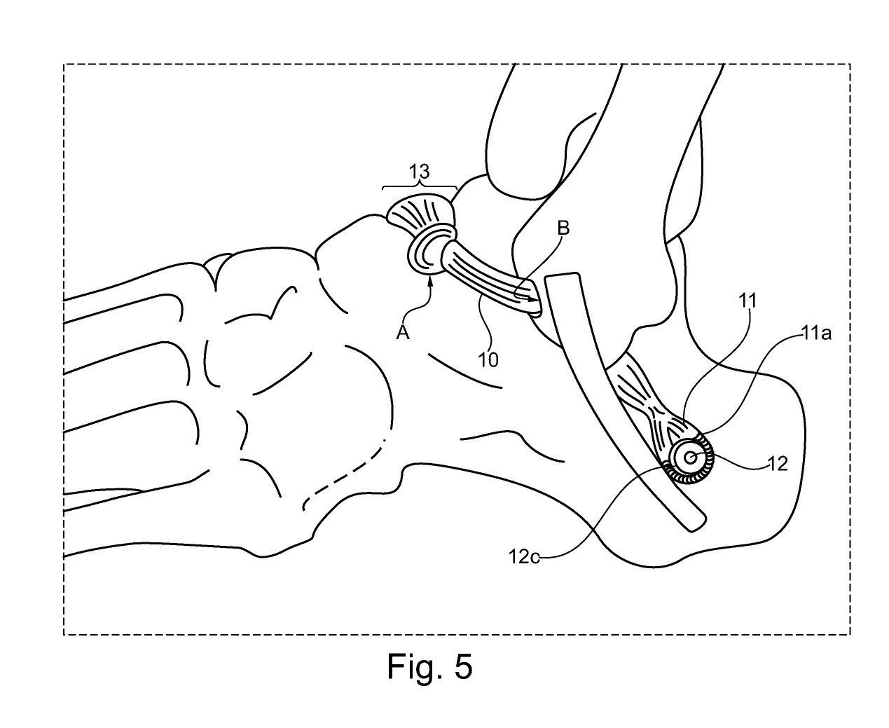

FIG. 5 is a close-up lateral view of the ankle with an artificial ligament fixed in place;

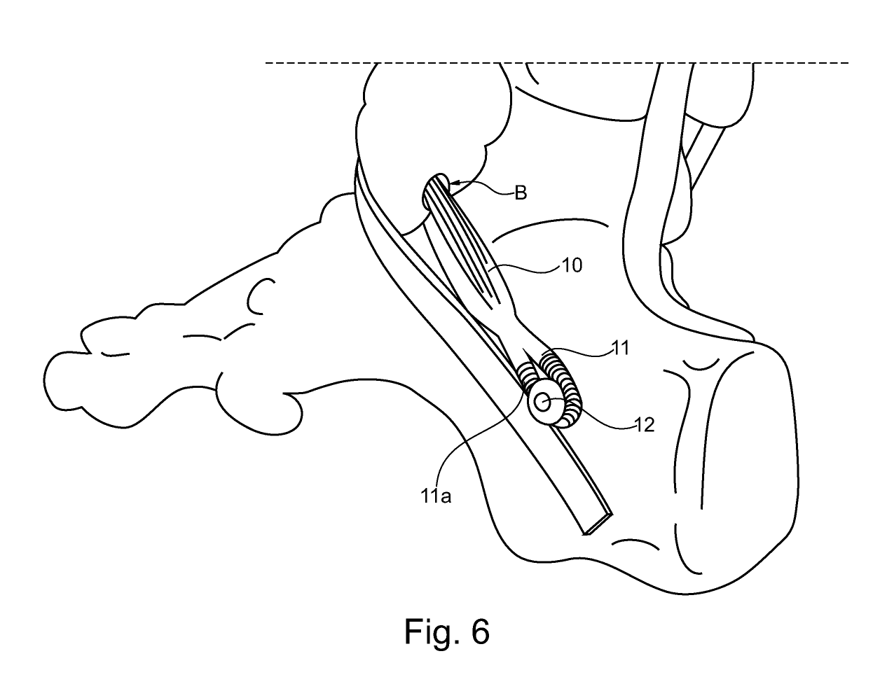

FIG. 6 is a posterior view of the bones of the ankle with an artificial ligament fixed in place;

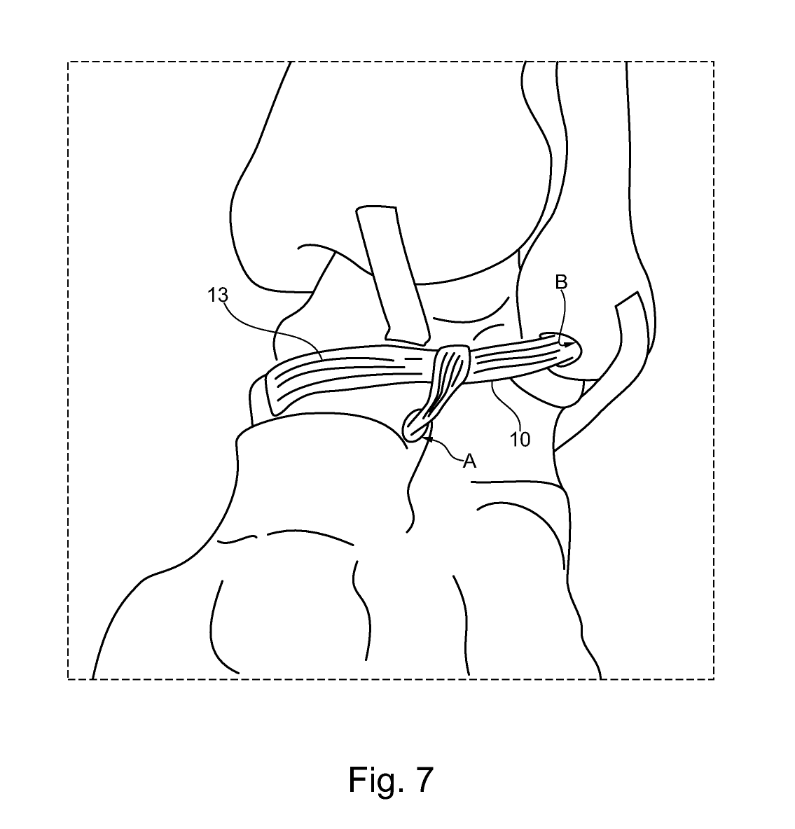

FIG. 7 is a close-up lateral view of the bones of the ankle with an artificial ligament fixed in place; and

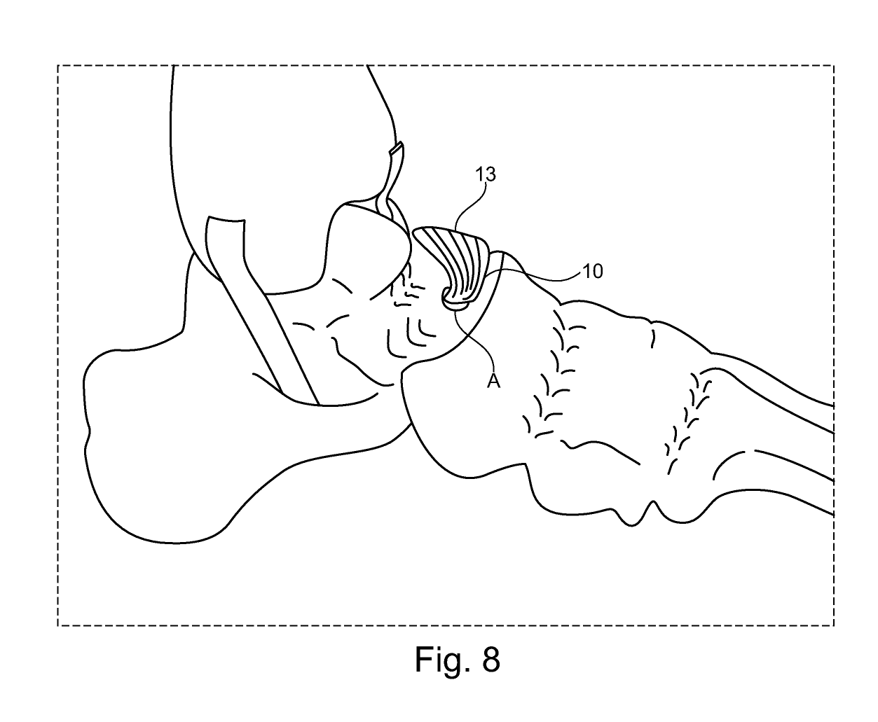

FIG. 8 is a medial view of the bones of the ankle with an artificial ligament fixed in place.

Referring to the drawings in general but particularly to FIGS. 4 to 8, in which there is shown an artificial ligament 10 implanted within the ankle in order to provide fixation during the healing process following trauma to the ligaments of the ankle. Whilst the ligament may take one of a number of forms, it has been found that a ligament having a hard loop 11 at a first end suitable for anchoring one end to solid bone material by means of a screw or bollard arrangement and a soft loop 13 at a second end suitable for securing to bone material by looping the length L of the ligament through the soft loop in a manner such as to anchor the soft loop end to the bone material is preferred for the procedure. Other forms of ligament such as ones having two hard loops may also be used, as may ligaments having other means for securing them in anchor holes or to bone material.

The procedure for the insertion of an artificial ligament 10 into an ankle joint comprises the following steps:
1. The ankle bones are exposed by making an incision in the region of the lateral malleolus (XX). Preferably, this is a longitudinal, ventrally curved skin incision just over the posterior margin of the lateral malleolus.
2. The neck NT of the talus T, the fibulotalar joint FJ and the subtalar joints SJ are identified, and a hole is then drilled in the direction of lines A-A through the talar neck using, for example, a 5.5 mm cannulated drill bit to create a bone tunnel (A).
3. An incision is then made on the medial side MS of the talus T adjacent to the bone tunnel (A).
4. A measuring device/length gauge for determining the length of the artificial ligament 10 to be used is then passed through the hole A in the talar neck, pulled through the medial incision, and fed back through the medial incision over the dorsal D part of the talar neck NT. In this regard, the measuring device MD would typically have a substantially similar structure as that of the artificial ligament and be made from similar material(s) with a metal leader (e.g. in the form of a metal wire) attached to one end of the measuring device. The measuring device is also provided with incremental markings that allow the correct length for the artificial ligament to be determined. The metal leader of the measuring device is then passed through an end loop of the measuring device MD and pulled taught around the talar neck.
5. Using the 5.5 mm cannulated drill bit, a fibula hole B is then drilled through the distal part of the fibula, in, preferably, the anteroposterior AP direction, approximately 7 mm proximal from the tip of the fibula, protecting the peroneal tendons PT. The measuring device is then passed through the fibula hole B.
6. Next, the position for a hard loop 11 location X of the artificial ligament 10 is prepared on the lateral wall LC of the calcaneus C, and the length of artificial ligament to be used is measured by stretching the measuring device to the correct spot and reading off the length from the graduations provided thereon. To determine the length of artificial ligament, the ankle joint and the subtalar joints are fully reduced under direct vision, by pulling on the measuring device and holding the foot in eversion and valgus. To get a better angle for reduction, the measuring device is pulled through a soft tissue canal towards the plantar side.
7. After obtaining the length measurement, the measuring device MD is removed from the fibula hole B, and the loop around the talus NT neck is undone.
8. The soft loop of the measuring device may then be passed through a hard loop 11 of the artificial ligament 10, and the artificial ligament 10 is pulled through the hole A of the talus neck and looped over the dorsal part of the talus neck. Alternatively, the artificial ligament may be passed through hole A by any other convenient means.
9. If the measuring device is being used, the metal leader of the measuring device is then passed through the soft loop 13 and the artificial ligament 10 which is then anchored over the talar neck by pulling the loop tight. A loop tensioner (not shown) may then be used to secure the ligament. Alternatively, the artificial ligament alone may be passed through the soft loop and pulled tight.
10. The measuring device with the artificial ligament 10 attached (or the ligament alone) is then fed through the fibula hole (B).
11. The artificial ligament 10 is then pulled tight and secured onto the calcaneus using a small fragment screw 12, bollard 12a and a washer 12c. Before fully tightening the screw, the measuring device (if used) is cut off the end of the hard loop (11). After a wash, the wound is closed in layers.

In the broadest application of the present invention there is provided a method for the insertion of an artificial ligament having a first end and as second end into the human or animal body comprising the steps of: drilling a first hole A through a first portion of bone material such as to provide an anchoring point, drilling a second hole B through a second, different, portion of bone material which it is desired to secure, securing the first end of the ligament to the anchor point, passing the second end of the ligament through said second hole and tensioning said ligament before securing said second end to a further portion of bone material separate from said first portion. The ligament may be provided having a soft loop at said first end and said ligament may then be secured to said anchor point by passing the second end of said ligament through said hole A and then passing said second end through the soft loop of said first end. Such an arrangement provides a loop anchor using the bone material as the anchor point. If the ligament is provided with a hard loop at said second end, said second end may be secured to said further portion of bone material by a fixation device passing through said hard loop. The securing of said hard loop end to said further portion of bone material may be by inserting a screw or bollard through said hard loop and securing said screw or bollard to said further portion of bone material. In the above process the first portion of bone material may be the talar neck T of an ankle, the second portion of bone material may be the distal part of the fibular and the further portion of bone material may be the calcaneus. Other locations for use of the method include the elbow, the knee and the wrist.

The artificial ligament 10 mentioned in the above described process preferably comprises a soft loop end 13 formed from the ligament material itself and formed in the manner of a twisted flat braided mesh having a width W greater than its depth D and being flexible enough to allow the tape to be formed into a loop having an opening O for receiving the hard loop end 11 through it such as to form an anchoring loop end comprising relatively soft and pliable material which presents little if any hard surface to irritate the patient and which can lie within the neck of the talus NT so as to produce a low profile anchoring point. The hard loop end 11 may comprise the same artificial ligament material as used on the soft loop end but it may be strengthened by binding the loop tightly with an over binding of similar material shown generally at 11a in FIG. 4. The tight over binding 11a helps provide a resistant surface for engagement with the screw 12 or washer 12c and prevents or reduces wear.

It will be appreciated that individual items described above may be used on their own or in combination with other items shown in the drawings or described in the description and that items mentioned in the same passage as each other or the same drawing as each other need not be used in combination with each other. In addition, the expression “means” may be replaced actuator or system or device as may be desirable. In addition, any reference to “comprising” or “consisting” is not intended to be limiting in any way whatsoever and the reader should interpret the description and claims accordingly. Furthermore, although the invention has been described in terms of preferred embodiments as set forth above, it should be understood that these embodiments are illustrative only. Those skilled in the art will be able to make modifications and alternatives in view of the disclosure which are contemplated as falling within the scope of the appended claims.

1. A method for the insertion of an artificial ligament into an ankle of a human, the method comprising:
drilling a first hole through the talar neck of the ankle;
drilling a second hole though the distal part of the fibular;
passing the artificial ligament through the first hole, looping the artificial ligament over the dorsal part of the talar neck and using a soft loop of the artificial ligament to anchor the artificial ligament over the talar neck;
passing the artificial ligament through the second hole and securing the artificial ligament onto the calcaneus using a screw inserted through a hard loop of the artificial ligament.

2. The method according to claim 1, further comprising:
after drilling the first hole and the second hole, using a measuring device that has been passed through the first hole and the second hole to determine the length of the artificial ligament to be used.

3. The method according to claim 2, wherein the measuring device is used to pull the artificial ligament through the first hole and the second hole.

4. The method according to claim 1, wherein the first hole is drilled using a cannulated drill bit.

5. The method according to claim 1, wherein the second hole is drilled through the distal part of the fibula, in the anteroposterior (AP) direction, approximately 7 mm proximal from the tip of the fibula.

6. The method according to claim 1, wherein the second hole is drilled using a cannulated drill bit.

7. The method according to claim 1, further comprising:
initially making a longitudinal, ventrally curved skin incision just over the posterior margin of the lateral malleolus.

8. The method according to claim 1, further comprising:
making an incision on the medial side of the talus adjacent to the first hole.

9. A kit of parts for use in the method of claim 1, comprising one or more artificial ligaments having an elongate portion, a first end including a loop for receiving a securing member and a second end including a loop for allowing said elongate portion to be passed therethrough such as to form a loop for securing to bone material and one or more securing members.

10. The kit of parts of claim 9, further comprising a plurality of artificial ligaments of different lengths.

11. The kit of parts of claim 9, further comprising a plurality of bone securing members in the form of screws or bollards.

12. The kit of parts of claim 9, wherein said one or more artificial ligaments comprise artificial ligaments having a first soft loop end and a second hard loop end.

13. The kit of parts of claim 9, further comprising a length gauge.

14. A method for the insertion of an artificial ligament having a first end and a second end into a human or animal body comprising the steps of: drilling a first hole through a first portion of bone material such as to provide an anchor point, drilling a second hole through a second, different, portion of bone material which it is desired to secure, securing the first end of the artificial ligament to the anchor point, passing the second end of the artificial ligament through said second hole and tensioning said artificial ligament before securing said second end to a further portion of bone material separate from said first portion and said second portion.

15. The method of claim 14, wherein the artificial ligament is provided having a soft loop at said first end and said artificial ligament is secured to said anchor point by passing the second end of said artificial ligament through said first hole and then passing said second end through the soft loop of said first end.

16. The method of claim 14, wherein the artificial ligament is provided with a hard loop at said second end and said second end is secured to said further portion of bone material by a fixation device passing through said hard loop.

17. The method of claim 16, further comprising securing said hard loop to said further portion of bone material by inserting a screw or bollard through said hard loop and securing said screw or bollard to said further portion of bone material.

18. The method of claim 14, wherein the first portion of bone material is the talar neck of the ankle, the second portion of bone material is the distal part of the fibular and the further portion of bone material is the calcaneus.
